(19) United States
(12) Patent Application Publication
(10) Pub. No.: US 2026/0256134 A1
(43) Pub. Date: Sep. 3, 2026

(54) NanoEmulsion Composite Mixture of Essential Oils with Biodegradable Polymers and Co-Surfactants for Antimicrobial Activities and Enhanced Thermal Stability and Antioxidant Activities

(71) Applicant: The American University in Cairo, New York, NY (US)

(72) Inventors: Wael Mamdouh Sayed Sayed Ahmed, Cairo (EG); Amro Abdel Azeem Hassan Shetta, Tanta (EG)

(21) Appl. No.: 18/878,852

(22) PCT Filed: Jun. 23, 2023
(86) PCT No.: PCT/US2023/026070

§ 371 Date: Mar. 4, 2025

Related U.S. Application Data
(60) Provisional application No. 63/355,213, filed on Jun. 24, 2022.

(51) Int. Cl. A01N 25/04; A01P 1/00

(52) U.S. Cl. CPC A01N 25/04; A01P 1/00

(57) ABSTRACT

A nanoemulsion composite mixture for antimicrobial activities is provided having biodegradable polymers, co-surf actants, surfactants and thickeners together entrapping an oil. The mixture is dissolved in glacial acetic acid and dichloromethane. Water is used as the vehicle to hold the mixture, and in the mixture: the biodegradable polymers are within 5-8%, the glacial acetic acid is within 0.5-1.5%, the oil is within 0.5-1.5%, the surfactant is within 0.5-1.5%, the thickener is within 15-25%, the dichloromethane is within 2.5-10%, and the co-surfactants is within 0.5-1.5%. These nanoemulsion composite mixture exhibits superior antimicrobial abilities, can kill 99.99% of different bacteria without causing any harmful side effect to humans. The nanoemulsion composite mixture can be incorporated in products like hand gel sanitizers, soap, detergents, animal feed, and more.

### FIELD OF THE INVENTION

This invention relates to nano-emulsion of oil mixtures with biodegradable polymers and co-surfactants.

### BACKGROUND OF THE INVENTION

Microbial infection is prevailing world-wide and is a high reason for mortality and morbidity leading to economic burden arising from both the decreased healthiness and the high cost spent on health care. Traditional disinfectants contain harsh chemical and alcohols, and have major problems related to dermal toxicities which may on the long run cause severe health issue such as asthma, lung cancer, skin cancer, eye infection and many others. The present invention advances the art with new nano-emulsion composites addressing at least some of these problems.

### SUMMARY OF THE INVENTION

The present invention provides a nanoemulsion composite mixture for antimicrobial activities.

The nanoemulsion composite mixture can be defined in three forms: comprising, consisting essentially of, or consisting of: biodegradable polymers, co-surfactants, surfactants and thickeners together entrapping an oil. The nanoemulsion composite mixture is dissolved in glacial acetic acid and dichloromethane, where water is used as the vehicle to hold the nanoemulsion composite mixture, and wherein in the nanoemulsion composite mixture: the biodegradable polymers are within 5-8%, the glacial acetic acid is within 0.5-1.5%, the oil is within 0.5-1.5%, the surfactant is within 0.5-1.5%, the thickener is within 15-25%, the dichloromethane is within 2.5-10%, and the co-surfactants is within 0.5-1.5%.

In one embodiment, the thickener is glycerin.

In another embodiment, the surfactant is Tween 80.

In yet another embodiment, the biodegradable polymers are Chitosan, 1,3 Propyleneglycolalginate, Gelatin, Pectin, 3-hydroxypentanoic acid, Glucose, Poly (acrylates), Albumin, Gluten, Poly (alkyl cyanoacrylate), Alginate, Glycerol and various, Poly (e-caprolactone), Alginic acid, Hydroxyethyl starch, poly (lactic-co-glycolic acid), Carrageenan, Hydroxypropylstarch, Polyanhydrides, Casein Cellulose, Lactic acid, Polylactic acid, Cellulose, Lactic acid, butyl ester, Polyphosphazenes, Cellulose acetate, Lignocellulose, polyvinyl alcohol, Cellulose acetate butyrate, Sucrose, Propyleneglycolalginate, Cellulose derivative, zein, starch, Chitin, Dextrin, Edible Starch, Hydrolysed Starch or Dextrin.

In yet another embodiment, the oil is Green tea essential oil, Ajwain Essential Oil, Almond Oil, Ambrette Seed Essential Oil, Angelica root Oil, Basil Essential oil, Bay oil, Bergamot oil, Black Pepper Oil, Black Seed essential oil, Cade oil, Cajeput oil, Calamus Essential Oil, Caraway seed, Carrot Seed Oil, Cassia Bark Oil, Castor Oil, Chamomile Essential Oil, Cinnamon essential oil, Citronella Essential Oil, Coconut Oil, Coriander Seed Oil, Cumin oil, Curry Leaf Oil, Cypress oil, Davana Essential Oil, Dill Seed Essential Oil, E. Citriodora Essential Oil, Sesame oil, Fennel Essential Oil, Fractionated Coconut Oil, Frankincense Essential Oil, Garlic Oil, Geranium Essential Oil, Ginger Essential Oil, Globulus Essential Oil, Grapefruit, Green tea essential oil, Holy Basil Essential Oil, Indian Rosemary Essential Oil, Jojoba Oil, Juniper Berry Oil, Karanj Oil, Lavender Oil, Lemongrass Essential Oil, Lemongrass Essential Oil, Lemongrass Essential Oil, Lime Oil, Mandarine, Marigold Essential Oil, Mentha arvensis Essential Oil, Mentha citrata Essential Oil, Mentha Peperata Essential Oil, Mentha Spicata Essential Oil, Moringa Seed oil, Nagarmotha Essential oil, Neem Oil, Nutmeg Essential Oil, Palmarosa Essential Oil, Patchouli Essential Oil, Peanut Oil, Sage Essential Oil, Soybean Oil, Spearmint, Sweet Marjoram oil, Tagetes minuta Essential Oil, Tamannu Essential Oil, Tea tree essential oil, Tea Tree Oil, Vetiver Essential Oil, Orange Oil, Thyme red Essential Oil, Wormwood, or Ylang Ylang Essential Oil.

In still another embodiment, the co-surfactants are Benzalkonium chloride, Behentrimonium chloride, Carbethopendecinium bromide, Didecyldimethylammonium chloride, Benzalkonium chloride, Cetalkonium chloride, Dimethyldioctadecylammonium bromide, Benzethonium chloride, Cetrimonium bromide, Dimethyldioctadecylammonium chloride, Benzododecinium bromide, Cetrimonium chloride, Dioleoyl-3-trimethylammonium propane, Bronidox, Cetylpyridinium chloride, Domiphen bromide, Lauryl methyl gluceth-10 hydroxypropyl dimonium chloride, Pahutoxin, Tetramethylammonium hydroxide, Octenidine dihydrochloride, Stearalkonium chloride, Thonzonium bromide, Olaflur, N-Oleyl-1,3-propanediamine, or Octenidine dihydrochloride.

### BRIEF DESCRIPTION OF THE DRAWINGS

FIG. 1 shows according to an exemplary embodiment of the invention a nano-emulsion system is composed of five different type of main components: Four components: biodegradable polymer such as chitosan, co-surfactant such as benzalkonium chloride, surfactants and thickeners. These four type of components enhance the entrapment of the fifth component type such as green tea essential oil.

FIGS. 2-4 show according to exemplary embodiments of the invention antimicrobial activities for the 6 formulations against 6 different microbial strains. Each block of bar graphs is in the order of formula 1 through 6 from left to right in each respective block of graph bars. For color interpretation of these graphs the reader is referred to the priority document of this application.

FIG. 5 shows according to an exemplary embodiment of the invention pour plate colony counting antimicrobial assay against S. aureus and E. coli.

FIG. 6 shows according to an exemplary embodiment of the invention improved essential oils thermal stability.

FIG. 7 shows according to an exemplary embodiment of the invention improved antioxidant activity.

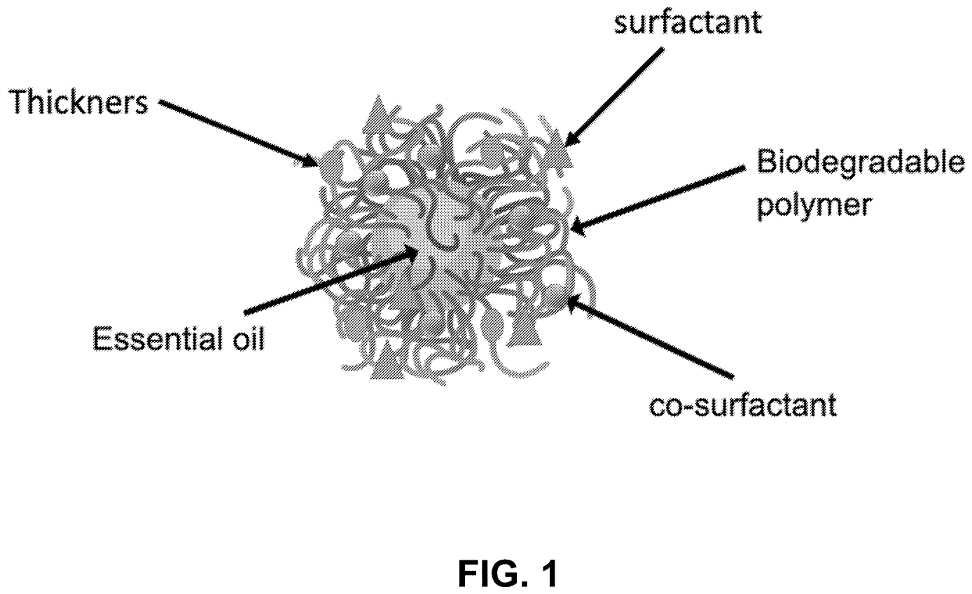

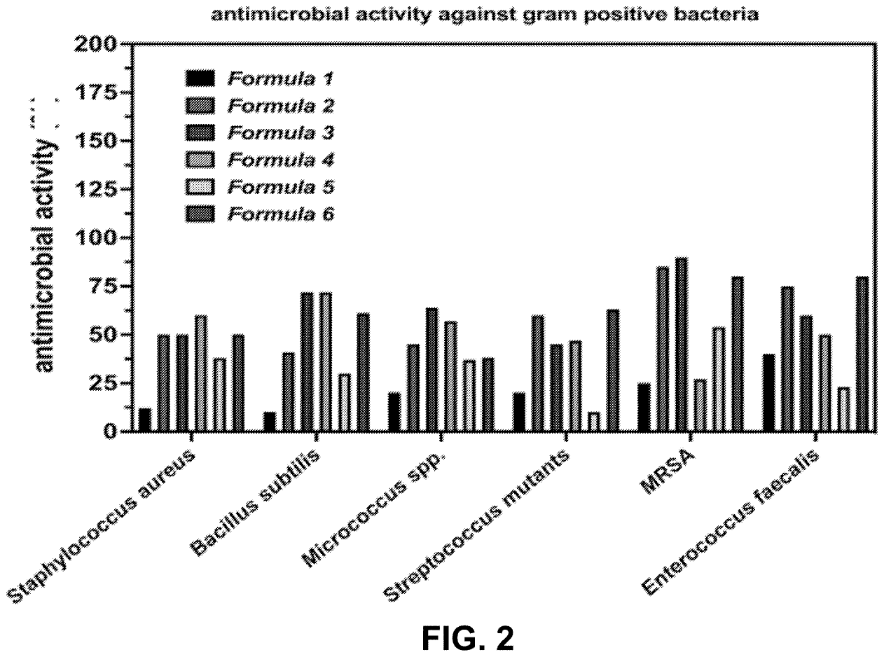

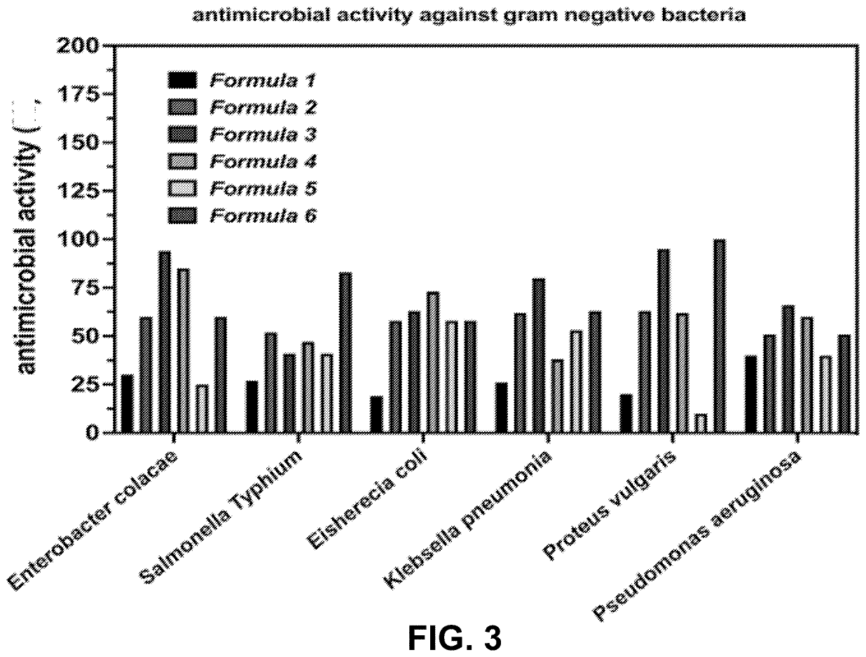

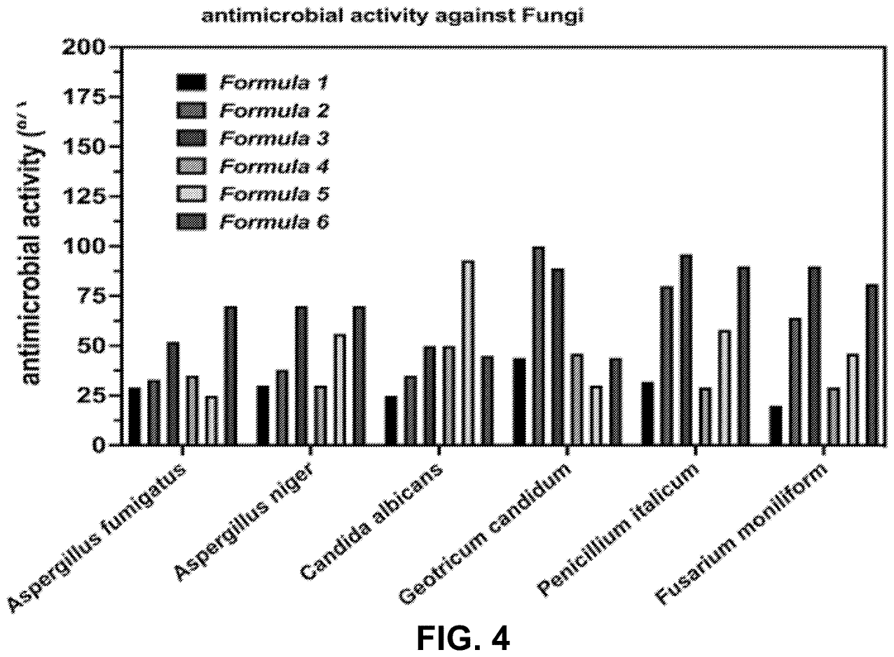

### DETAILED DESCRIPTION

The objective of the present invention was to develop a natural and eco-friendly mixture of oils (e.g. fixed oils, mineral oils, oleo-resin oils, and essential oils) and biodegradable polymers and mixing all of them (with different components, not limited to, such as: co-surfactants and/or viscosity modifiers and/or stabilizers and/or pH adjusters and/or humectants and/or moisturizing and/or preservatives and/or chelating agents and/or buffering agents and/or skin conditioning and/or solvents and/or antioxidants) by using conventional and mixers and/or high speed homogenizers under certain speed and duration, to have a final product as a natural disinfectant/antiseptic/antioxidant with broad antimicrobial activities and skin regeneration and cleansing activities.

The mixing step of all ingredients determines the final product, for example, either composite mixture of all ingredients with mixers and/or nanoscale transformation of the composite mixture by high-speed homogenizers. The latter makes the mixture exhibiting superior improvement in the antibacterial/disinfectant/antioxidant properties compared to their composite bulk solutions and can be prepared as nanoparticles and nano-emulsions, nano-suspensions and solubilized preparations.

The nanoemulsion composite mixture can be prepared as liquid solution and gel and powder additive forms to be used as natural disinfectant/antiseptic/antioxidant, and can also be incorporated in all cosmetics products, pesticides and animal feed, food beverages to enhance their antimicrobial and antioxidant and preservation properties while maintaining their natural flavor and function. Table 1 shows a composition of key components of the nano-emulsion composition.

TABLE 1
A list of key components of the nano-emulsion composition.
Each 1 L contains:

| S.N. | Ingredient name | Quantity | | Function | Specification |
|---|---|---|---|---|---|
| 1- | Chitosan | 65 | g | Biodegradable polymer with | USP 40 |
| | | | | two functions: Co-surfactant | |
| | | | | and antiseptic | |
| 2- | Glacial acetic acid | 10 | g | Solvent | |
| 3- | Green tea essential oil | 10 | g | Solvent | |
| 4- | Tween 80 | 11.2 | g | Surfactant | |
| 5- | Glycerin | 200 | g | Thickener | |
| 6- | Dichloromethane | 50 | g | Solvent | |
| 7- | Benzalkonium chloride | 10 | g | Co-surfactant and antiseptic | |
| 8- | Distilled water | 1000 | gm | Vehicle | USP40 |

These different components added to the nano-emulsion formula each playing different roles. The following components are key components, yet some components could be varied as will be discussed infra.
Benzalkonium chloride is a quaternary ammonium antiseptic and disinfectant with actions and uses similar to those of other cationic surfactants. It can also be used as an antimicrobial preservative for pharmaceutical products.
Glacial acetic acid is the anhydrous (undiluted or free of water) form of acetic acid. Acetic acid is considered an organic compound and has the chemical formula CH₃COOH. A diluted solution of acetic acid is known as vinegar or ethanoic acid or ethylic acid. This acid is classified as a weak acid, and it is used as a solvent to dissolve chitosan molecules.
Green tea essential oil is an anti-free radical, an antioxidant and a skin regenerating (anti-wrinkles, anti-ageing) as well as their ability of killing microorganisms due to their phenolic contents.
Tween 80 (polysorbate 80, polyoxyethylene sorbitan monooleate) is a non-ionic surfactant that is widely used as an emulsifier both in the laboratory and in industry. It is often included in laboratory experiments without any comments or evaluations of possible side effects.
The presence of glycerol in an oil-in-water emulsion will increase the solubility of the non-polar oil molecules in the surrounding aqueous phase since a glycerol solution is less polar than pure water.
Dichloromethane is a clear colorless, volatile, sweet-smelling lipophilic liquid. It is commonly used as a solvent of green tea essential oil.
Chitosan is a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, such as sodium hydroxide. Chitosan has fascinating antibacterial activity, good biodegradation, outstanding biocompatibility, non-toxicity and excellent physical and chemical properties. As a result, chitosan has been widely used in the field of antibacterial. Chitosan and its derivatives show antibacterial activity against fungi, gram-positive bacteria and gram-negative bacteria. In recent years, there have been some reviews about the antibacterial activity of chitosan and its derivatives.

The mixing step of all ingredients determines the final shape of the product, for example, either composite mixture of all ingredients with mixers and/or nanoscale transformation of the composite mixture by high-speed homogenizers. Briefly, chitosan solution (1% w/v) is prepared after dissolving chitosan powder in glacial acetic acid (1% w/v). Then, Tween 80 is stirred with chitosan solution forming the aqueous phase of the emulsion. In dichloromethane, green tea essential oil is dissolved forming the oily phase of the emulsion. On the semi-industrial scale rotor-stator homogenizer, in an exemplary embodiment, the oily phase is homogenized with the aqueous phase at 3000 rpm for 20 min to get an oil in water emulsion. Then benzalkonium chloride, glycerin and distilled water are added step wise forming the antiseptic nanoemulsion composite mixture.

The latter makes the nanoemulsion composite mixture exhibiting superior improvement in the antibacterial, disinfectant, antioxidant properties compared to their composite bulk solutions.

The final forms of the mixed products (nanoemulsion composite mixture) can be prepared as liquid solution and gel and powder additive forms that to be used as natural disinfectant/antiseptic/antioxidant, and can also be incorporated in all cosmetics products, and added to all food beverages to enhance their antimicrobial and antioxidant and presentation properties while maintaining their natural flavor and function and can also be added as additives to pesticides and animal feed. The nanoemulsion composite mixture showed:
(i) Enhanced essential oils water solubility
(ii) Improved essential oils thermal stability
(iii) Boosted the antioxidant activity of essential oil
(iv) Improved in the microbial activities of essential oils against both Gram-positive, Gram-negative bacteria, fungi, and viruses.

### Enhanced Essential Oils Water Solubility

Nano-emulsions constitute one interesting vehicle for enhancing solubility, stability and delivering natural oils, by encapsulating them into nanosized micelles with sizes ranging from 20-200 nm. They gather some unique characteristics as small size, increased surface area and stability which can increase efficiency and biological effects of pharmaceutical dosage forms.

### Improved Essential Oils Thermal Stability

Thermogravimetric analysis (TGA) analysis is performed to study the thermal stability of green tea essential oil, after encapsulation. The temperature at which a material is subjected to the highest rate of weight loss is known as the temperature of maximum degradation rate (Td) TGA studies showed a significant improvement of the thermal stability of nanoencapsulated green tea essential oil by about 1.74 folds (or in another embodiment more than 1.5 folds) over the bulk green tea essential oil.

### Boosted the Antioxidant Activity of Essential Oil

DPPH radical scavenging ability is a method that used to estimate the antioxidant properties of the was identified for the formula. Gallic acid was used as positive control. Surprisingly the antioxidant activity of green tea essential oil is increased significantly by about 2-fold (or in another embodiment more than 2-folds) after nanoencapsulation process with IC50 (concentration required to scavenge DPPH radicals by 50%) of 0.34 mg/mL (or in another embodiment more than 0.3 mg/mL).

### Hypoallergic Feature

The most updated animal test results showed that the nanoemulsion composite mixture samples didn't show any sensitivity reactions on the skin on three different rabbits that are used in this trials testing. Accordingly, the nanoemulsion composite mixture samples showed conformance with requirements according to the tests mentioned in the Egyptian standards.

### Improved the Antimicrobial Activities of Essential Oils Against Both Gram-Positive, Gram-Negative Bacteria and Fungi

Inhibition zone method was followed in order to test the antimicrobial efficiency of the selected nanoemulsion composite mixture samples against 6 chosen strains of gram-positive bacterial strains, 6 strains of gram-negative bacterial strains 6 strains of fungi as shown in the following list;

### 1-Gram-Positive Bacterial:

| | |
|---|---|
| a) | Staphylococcus aureus |
| b) | Bacillus subtilis |
| c) | Micrococcus spp. |
| d) | Streptococcus mutants |
| e) | Methicillin-resistant Staphylococcus aureus (MRSA) |
| f) | Enterococcus faecalis |

### 2-Gram-Negative Bacteria:

| | |
|---|---|
| a) | Enterobacter colacae |
| b) | Salmonella Typhium |
| c) | Eisherecia coli |
| d) | Klebsella pneumonia |
| e) | Proteus vulgaris |
| f) | Pseudomonas aeruginosa |

### 3-Fungi:

| | |
|---|---|
| a) | Aspergillus fumigatus |
| b) | Aspergillus niger |
| c) | Candida albicans |
| d) | Geotricum candidum |
| e) | Penicillium italicum |
| f) | Fusarium moniliform |

The nanoemulsion composite mixture was tested to find out the percentage of dilution that can stop the growth of microbes. This method is known as (MIC=minimum inhibitory concentration). At the time, the results showed that the sample can stop the growth of microbes even after 13 times of dilution with some types of microbes such as staph. mutants. Sample showed antimicrobial activities with an ability close to commercial preparations containing strong chemicals or alcohols especially against Geo. Candidium, staph. mutants, B. subtilis, Entero. faecllialis, and E. colace. Moreover, sample that proved effectiveness were also tested to see if the samples had the ability to kill microbes (bactericidal effect=cidal) or could only stop their growth (bacteriostatic effect=static). This method is known as (MIC=minimum toxic concentration). As shown in (Table 2), The results showed that the nanoemulsion composite mixture samples can kill different types of microbes, and in other cases, they were only able to stop the growth of some microbes.

TABLE 2
The antimicrobial activity of the nanoemulsion composite mixture
against gram-positive, gram-negative bacteria and fungi.

| | | Nanoemulsion composite |
|---|---|---|
| Microbial strains | | mixture |
| Gram-positive | Staphylococcus aureus | Bactericidal |
| bacteria | Bacillus subtilis | Bacteriostatic |
| | Micrococcus spp. | Bacteriostatic |
| | Streptococcus mutants | Bacteriostatic |
| | MRSA | Bacteriostatic |
| | Enterococcus faecalis | Bacteriostatic |
| Gram-negative | Enterobacter colacae | Bacteriostatic |
| bacteria | Salmonella Typhium | Bactericidal |
| | Klebsella pneumonia | Bactericidal |
| | Proteus vulgaris | Bactericidal |
| | Pseudomonas aeruginosa | Bactericidal |
| Fungi | Aspergillus fumigatus | Bactericidal |
| | Aspergillus niger | Bactericidal |
| | Candida albicans | Bactericidal |
| | Geotricum candidum | Bactericidal |
| | Penicillium italicum | Bactericidal |
| | Fusarium moniliform | Bactericidal |

### Improved the Antimicrobial Activities of Essential Oils Against COVID-19

The antiviral activity for the all the nanoemulsion composite mixture samples was performed at Center of Scientific Excellence for Influenza Viruses and Consultant Unit for Viruses Research and Bioassays at National Research Centre (NRC). The nanoemulsion composite mixture samples shows high cytotoxic value for COVID-19. The nanoemulsion composite mixture samples show antiviral activity that reaches almost 100% with addition of harsh chemicals. Specifically, after the addition of quaternary ammonium compounds. The IC50 value is 0.0167 μL (or in another embodiment less than 0.05 μL).

Alternatively, the invention can be described as a composite Mixture of Essential Oils with biodegradable polymers and co-surfactants for broad antimicrobial activities and enhanced thermal stability and antioxidant activities. Mixing procedure determines the final compositional structure of nanoemulsion composite mixture. For the later, the following are the key aspects in the preparation:
1—Mixing all components, as for example shown in Table 1, into nano-emulsion formulation is based on high-speed Homogenization process with mechanical stirring range 300-30000 rpm.
2—Controlling the preparation parameters such as (i) homogenization speed (1000-3000 rpm) and homogenization time (10 min-30 min).
3—Controlling the solution parameters such as the selection of the desired essential oil(s), polymer and co-surfactant as well as their concentration as described in Table 7.
4—Identifying the antioxidant activities of the nanoemulsion composite mixture.
5—Investigating the antibacterial activities and determination of both minimum bactericidal concentration (MBC) and minimum inhibitory concentration (MIC) of the prepared nanoemulsion composite mixture against different gram-positive and gram-negative bacteria, viruses and fungi.
6—Investigating the thermal stability of the nanoemulsion composite mixture.
7—Estimating the IC50 of the nanoemulsion composite mixture against covid-19 (microbial) strains
8—Investigating the cytotoxicity of the nanoemulsion composite mixture in-vitro and on the skin of different animals (to study skin irritation as well).
9—Estimating the antimicrobial contact time needed for the In-vitro release of the active components (i.e. essential oils) from the nanoemulsion composite mixture at different pH values.
10—Studying the chemical stability of the essential oils after nanoencapsulation
11—Validation of the nanoemulsion composite mixture by testing at different industrial sites

The innovation is not limited to green tea essential oil, and it can include the following essential oils that are listed in Table 3.

TABLE 3
List of other Essential oils
List of Essential Oils

| | | |
|---|---|---|
| Ajwain Essential Oil | Almond Oil | Ambrette Seed Essential Oils |
| Angelica root Oil | Basil Essential oil | Bay oil |
| Bergamot oil | Black Pepper Oil | Black Seed essential oil |
| Cade oil | Cajeput oil | Calamus Essential Oil |
| Caraway seed | Carrot Seed Oil | Cassia Bark Oil |
| Castor Oil | Chamomile Essential Oil | Cinnamon essential oil |
| Citronella Essential Oil | Coconut Oil | Coriander Seed Oil |
| Cumin oil | Curry Leaf Oil | Cypress oil |
| Davana Essential Oil | Dill Seed Essential Oil | E. Citriodora Essential Oil |
| Sesame oil | Fennel Essential Oil | Fractionated Coconut Oil |
| Frankincense Essential Oil | Garlic Oil | Geranium Essential Oil |
| Ginger Essential Oil | Globulus Essential | Grapefruit |
| Green tea essential oil | Holy Basil Essential Oil | Indian Rosemary Essential Oil |
| Jojoba Oil | Juniper Berry Oil | Karanj Oil |
| Lavender Oil | Lemongrass Essential Oil | Lemongrass Essential Oil |
| Lemongrass Essential Oil | Lime Oil | Mandarine |
| Marigold Essential Oil | Mentha Arvensis Essential Oil | Mentha Citrata Essential Oil |
| Mentha Peperata Essential Oil | Mentha Spicata Essential Oil | Moringa Seed oil |
| Nagarmotha Essential oil | Neem Oil | Nutmeg Essential Oil |
| Palmarosa Essential Oil | Patchouli Essential Oil | Peanut Oil |
| Sage Essential Oil | Soybean Oil | Spearmint |
| Sweet Marjoram oil | Tagetes Minuta Essential Oil | Tamannu Essential Oil |
| tea tree essential oil | Tea Tree Oil | Vetiver Essential Oil |
| Orange Oil | Thyme red Essential Oil | Wormwood |
| Ylang Ylang Essential Oil | | |

The innovation is not limited to chitosan, and it can include the following biodegradable polymers that are listed in Table 4.

TABLE 4
List of Other Biodegradable polymers
List of biodegradable polymers

| | | |
|---|---|---|
| 1,3 Propyleneglycolalginate | Gelatin | Pectin |
| 3-hydroxypentanoic acid | Glucose | Poly (acrylates) |
| Albumin | Gluten | Poly (alkyl cyanoacrylate) |
| Alginate | Glycerol and various | Poly (e-caprolactone) |
| Alginic acid | Hydroxyethyl starch | poly(lactic-co-glycolic acid) |
| Carrageenan | Hydroxypropylstarch | Polyanhydrides |
| Casein Cellulose | Lactic acid | Polylactic acid |
| Cellulose | Lactic acid, butyl ester | Polyphosphazenes |
| Cellulose acetate | Lignocellulose | polyvinyl alcohol |
| Cellulose acetate butyrate | Sucrose | Propyleneglycolalginate |
| Cellulose dervatives, | zein | starch |
| Chitin | Dextrin | Starch, edible |
| Dextrin | Starch, hydrolysed | |

The innovation is not limited to benzalkonium chloride, and it can include the following co-surfactants that are listed in Table 5.

TABLE 5
List of Other Co-surfactants
List of co-surfactants

| | | |
|---|---|---|
| Behentrimonium chloride | Carbethopendecinium | Didecyldimethylammonium chloride |
| | bromide | |
| Benzalkonium chloride | Cetalkonium chloride | Dimethyldioctadecylammonium |
| | | bromide |
| Benzethonium chloride | Cetrimonium bromide | Dimethyldioctadecylammonium |
| | | chloride |
| Benzododecinium | Cetrimonium chloride | Dioleoyl-3-trimethylammonium |
| bromide | | propane |
| Bronidox | Cetylpyridinium chloride | Domiphen bromide |
| Lauryl methyl gluceth-10 | Pahutoxin | Tetramethylammonium hydroxide |
| hydroxypropyl dimonium | | |
| chloride | | |
| Octenidine | Stearalkonium chloride | Thonzonium bromide |
| dihydrochloride | | |
| Olaflur | N-Oleyl-1,3- | Octenidine dihydrochloride |
| | propanediamine | |

The nanoemulsion composite mixture with exemplary formula components is preferably used with percentages and amount ranges as shown in Table 6. It is noted that these ranges and percentages apply to other examples discussed herein for the biodegradable polymers, oils and co-surfactants.

TABLE 6
Percentage and amount ranges of the formula components
Each 1 L contains

| | Quantity range | | | | Percentage range (%) | | |
|---|---|---|---|---|---|---|---|
| Ingredient name | Minimum | | Maximum | | Minimum | Maximum | Function |
| Chitosan USP 40 | 50 | g | 80 | g | 5 | 8 | Biodegradable |
| | | | | | | | polymer with two |
| | | | | | | | functions: Co- |
| | | | | | | | surfactant and |
| | | | | | | | antiseptic |
| Glacial acetic acid | 5 | g | 15 | g | 0.5 | 1.5 | Solvent |
| Green tea | 5 | g | 15 | g | 0.5 | 1.5 | Solvent |
| Tween 80 | 5 | g | 15 | g | 0.5 | 1.5 | Surfactant |
| Glycerin | 150 | g | 250 | g | 15 | 25 | Thickener |
| Dichloromethane | 25 | g | 100 | g | 2.5 | 10 | Solvent |
| Benzalkonium | 5 | g | 15 | g | 0.5 | 1.5 | Co-surfactant and |
| chloride | | | | | | | antiseptic |
| Distilled water | 1 | L | 1 | L | 100 | 100 | Vehicle |
| USP 40 to | | | | | | | |

Antimicrobial activities of the different formulations against 18 different microbial strains were studied as shown in Table 7. The following formulae were defined:
Formula 1 (F1): Green tea essential oil
Formula 2 (F2): Chitosan solution
Formula 3 (F3): benzalkonium solution
Formula 4 (F4): control (antimicrobial compound)
Formula 5 (F5): Green tea essential oil+Chitosan solution
Formula 6 (F6): The final formula of the nanoemulsion composite mixture

TABLE 7
Antimicrobial activity of the 6 formulations
expressed as % inhibition

| | | F1 | F2 | F3 | F4 | F5 | F6 |
|---|---|---|---|---|---|---|---|
| Gram-positive | Staphylococcus aureus | 12 | 50 | 50 | 60 | 38 | 50 |
| bacteria | Bacillus subtilis | 10 | 41 | 72 | 72 | 30 | 61 |
| | Micrococcus spp. | 20 | 45 | 64 | 57 | 37 | 38 |
| | Streptococcus mutants | 20 | 60 | 45 | 47 | 10 | 63 |
| | MRSA | 25 | 85 | 90 | 27 | 54 | 80 |
| | Enterococcus faecalis | 40 | 75 | 60 | 50 | 23 | 80 |
| Gram-negative | Enterobacter colacae | 30 | 60 | 94 | 85 | 25 | 60 |
| bacteria | Salmonella Typhium | 27 | 52 | 41 | 47 | 41 | 83 |
| | Eisherecia coli | 19 | 58 | 63 | 73 | 58 | 58 |
| | Klebsella pneumonia | 26 | 62 | 80 | 38 | 53 | 63 |
| | Proteus vulgaris | 20 | 63 | 95 | 62 | 10 | 100 |
| | Pseudomonas | 40 | 51 | 66 | 60 | 40 | 51 |
| | aeruginosa | | | | | | |
| Fungi | Aspergillus fumigatus | 29 | 33 | 52 | 35 | 25 | 70 |
| | Aspergillus niger | 30 | 38 | 70 | 30 | 56 | 70 |
| | Candida albicans | 25 | 35 | 50 | 50 | 93 | 45 |
| | Geotricum candiaum | 44 | 100 | 89 | 46 | 30 | 44 |
| | Penicillium italicum | 32 | 80 | 96 | 29 | 58 | 90 |
| | Fusarium moniliform | 20 | 64 | 90 | 29 | 46 | 81 |

Inhibition zone method was followed in order to test the antimicrobial efficiency of the 6 selected nanoemulsion composite mixture samples against 6 chosen strains of gram-positive bacterial strains, 6 strains of gram-negative bacterial strains 6 strains of fungi.

It is found that some of the selected samples have high antibacterial efficiency against most of the chosen bacterial strains when compared to the control such as MRSA and Enterobacter faecalis. Interestingly, it was found that some samples show good antibacterial activity even against highly resistant bacterial strains such as MRSA.

It is found that some of the selected samples have high antibacterial efficiency against most of the chosen bacterial strains when compared to the control. Interestingly, it was found that the two samples possess high antibacterial activity that is equal or greater than that of the control when tested with specific strains such as Salmonella typhium, Proteus vulgaris, and Pseudomonas aeruginosa. Moreover, It is found that some of the selected samples have high antifungal efficiency against most of the chosen bacterial strains when compared to the control. Interestingly, it was observed that those two samples showed good antimicrobial results against gram-positive bacteria, gram-negative bacterial and fungi. Therefore, they could be considered as promising antimicrobial sanitizers against wide range of microorganisms. It should be mentioned that the benzalkonium chloride (BAC) solution showed the highest antimicrobial activity against most of the microbial strains however, many studies have shown the harmful effects of BAC on the human body, such as skin irritation and allergic contact dermatitis. Highly toxic effects of BAC have also been found in animal studies. BAC orally administered to rats causes significant lethality, and the lethal dose 50 (LD50) has been reported to be 234-525 mg/kg.

### Stability Testing of the Optimum Formula

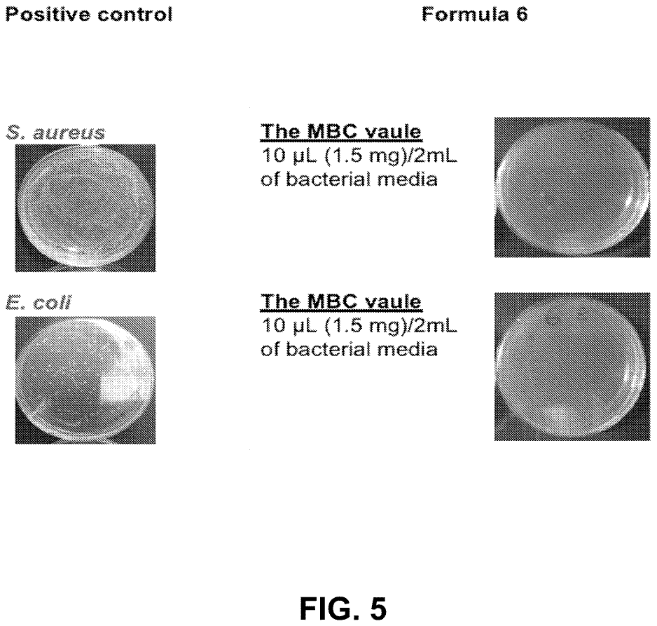

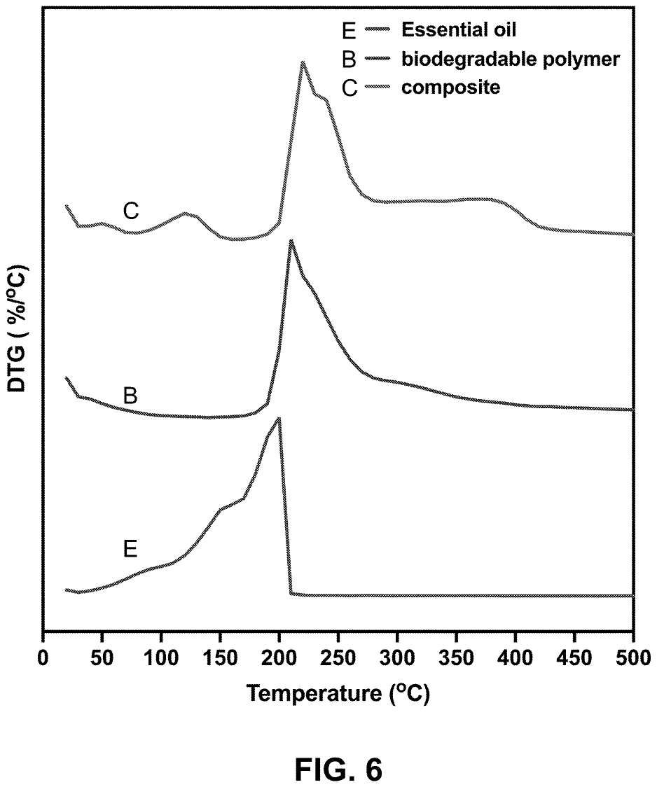

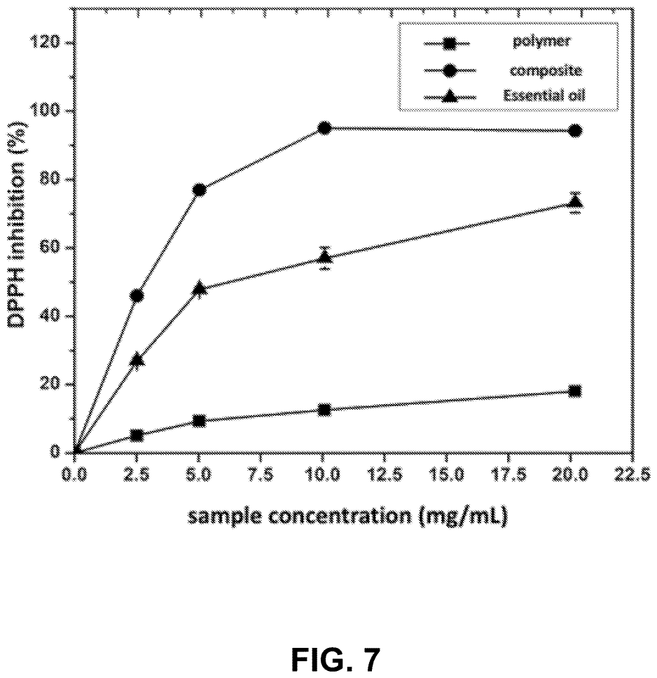

The stability for the nanoemulsion composite mixture formula was studied using the visual, DLS analysis as well as SEM analysis (FIG. 5). The results showed a good stability over one-month storage. According to the pH impact on the different nano-systems, the acidic media negatively affected the stability of the formula and induce their degradation. Thermogravimetric analysis (TGA) showed a significant improvement of the thermal stability of encapsulated essential oil 1.74 folds over the pure non-capsulated bulk essential oil. The thermal degradation of the essential oil occurred at temperature above 350° C.

TABLE 8
Main differences between nano-emulsions and nanoparticles

| | Nanoparticles | Nanoemulsions |
|---|---|---|
| Definition | Nanoparticles are small or ultrafine | Emulsion with droplet (referred to |
| | particles in a nanoscale range | as micelles) size in the nanoscale |
| | (normally between 1-100 nm) that | range (normally between 20-200 |
| | could be in Solid, liquid, or gas states | nm) that in a liquid state |
| Formation | The preparation of nanoparticles is | The preparation of nanoemulsion is |
| | achieved either by: | achieved by emulsification |
| | (i) Bottom-up approaches such as | techniques such as high-pressure |
| | chemical synthesis, precipitation, or | homogenization, sonication, |
| | self-assembly | microfluidization, and phase |
| | (ii)Top-down approaches such as | inversion. |
| | milling, grinding, or lithography | |
| Composition | Comprises various materials, | Comprises of oil nanosized droplets |
| | including metals, polymers, lipids, | in dispersed in water or water |
| | silica, or quantum dots | nanosized droplets dispersed in oil |
| Stability | nanoparticles are susceptible to | nanoemulsion exhibit enhanced |
| | degradation, sedimentation, and | stability due to small droplet size |
| | agglomeration | and presence of co-surfactants that |
| | | stabilizes the emulsion and prevent |
| | | coalescence of droplets |
| Drug | nanoparticles can encapsulate drugs | Nanoemulsion can encapsulate |
| Encapsulation | within the nanoparticle matrix | drugs within the oil or water |
| | | droplets |
| Drug Release | The drug release from nanoparticles | The drug release can be controlled |
| | can be controlled via modifying the | from nanoemulsion via modifying |
| | particle surface, matrix, or through | the emulsion composition, droplet |
| | stimulus-responsive systems | characteristics, or stimuli- |
| | | responsive systems |
| Toxicity | The toxicity of nanoparticles depends | The toxicity of nanoemulsions is |
| | on their composition and surface | primarily determined by the |
| | properties | components used, such as |
| | | surfactants or co-surfactants |

### Advantages

Using Nano-Encapsulation Technology to produce Eco-friendly disinfectants without any harsh chemicals or harmful materials by using natural materials which play a crucial role in enhancing the penetration and disruption of microbial membranes that cause inhibition effect on cell functional properties and eventually causing leakage of the internal contents of the microbial cell leading to death of the microbe. These novel nanoemulsion composite mixture exhibit superior antimicrobial abilities, can kill 99.99% of different bacteria without causing any harmful side effect to humans. These nanoemulsion composite mixtures can be incorporated in many products (hand gel sanitizers, soap, detergents, animal feed, and many more).

The unique power of Nano-Encapsulation Technology that it offers the ability to work near the atomic level to create structures with fundamentally new and superior beneficial physical properties and behaviors compared to any other material. The different nanoemulsion composite mixture produced are non-toxic, and controllable. They can be used in different forms (solution, suspension, emulsion, gel and powder). More importantly, we do not use any harsh chemicals or alcohol in our products, thus we offer the first of its kind Eco-friendly nanocapsulation technology-based disinfectant. More interestingly, the formulations can be added to any existing products (beverages, detergents, textile, cosmetics, pesticides, animal feed, etc).

The innovative nanoemulsion composite mixture could be added as a liquid additive with a percentage that ranges between 0.05-30% on the beverages, detergents, textile, cosmetics, pesticides, animal feed products.
1—Broad spectrum antimicrobial effect
2—Close antimicrobial effect compared to commercially available chemical-based disinfectants.
3—FDA approved and EPA registered ingredients.
4—Non-toxic, Biodegradable and eco-friendly
5—hypoallergenic
6—Powerful antioxidant properties
7—Long lasting protection.
8—Different forms (liquid, spray, soaps, powder)
9—High thermal stability of the natural components
10—High water stability
11—Works on both nonporous and porous surfaces
12—used both indoors and outdoors.

What is claimed is:

1. A nanoemulsion composite mixture for antimicrobial activities, comprising: biodegradable polymers, co-surfactants, surfactants and thickeners together entrapping an oil,
wherein the nanoemulsion composite mixture is dissolved in glacial acetic acid and dichloromethane,
wherein water is used as the vehicle to hold the nanoemulsion composite mixture, and
wherein in the nanoemulsion composite mixture:
the biodegradable polymers are within 5-8%,
the glacial acetic acid is within 0.5-1.5%,
the oil is within 0.5-1.5%,
the surfactant is within 0.5-1.5%,
the thickener is within 15-25%,
the dichloromethane is within 2.5-10%, and
the co-surfactants is within 0.5-1.5%.

2. The nanoemulsion composite mixture as set forth in claim 1, wherein the thickener is glycerin.

3. The nanoemulsion composite mixture as set forth in claim 1, wherein the surfactant is Tween 80.

4. The nanoemulsion composite mixture as set forth in claim 1, wherein the biodegradable polymers are Chitosan, 1,3 Propyleneglycolalginate, Gelatin, Pectin, 3-hydroxypentanoic acid, Glucose, Poly (acrylates), Albumin, Gluten, Poly (alkyl cyanoacrylate), Alginate, Glycerol and various, Poly (e-caprolactone), Alginic acid, Hydroxyethyl starch, poly (lactic-co-glycolic acid), Carrageenan, Hydroxypropylstarch, Polyanhydrides, Casein Cellulose, Lactic acid, Polylactic acid, Cellulose, Lactic acid, butyl ester, Polyphosphazenes, Cellulose acetate, Lignocellulose, polyvinyl alcohol, Cellulose acetate butyrate, Sucrose, Propyleneglycolalginate, Cellulose derivative, zein, starch, Chitin, Dextrin, Edible Starch, Hydrolysed Starch or Dextrin.

5. The nanoemulsion composite mixture as set forth in claim 1, wherein the oil is Green tea essential oil, Ajwain Essential Oil, Almond Oil, Ambrette Seed Essential Oil, Angelica root Oil, Basil Essential oil, Bay oil, Bergamot oil, Black Pepper Oil, Black Seed essential oil, Cade oil, Cajeput oil, Calamus Essential Oil, Caraway seed, Carrot Seed Oil, Cassia Bark Oil, Castor Oil, Chamomile Essential Oil, Cinnamon essential oil, Citronella Essential Oil, Coconut Oil, Coriander Seed Oil, Cumin oil, Curry Leaf Oil, Cypress oil, Davana Essential Oil, Dill Seed Essential Oil, E. citriodora Essential Oil, Sesame oil, Fennel Essential Oil, Fractionated Coconut Oil, Frankincense Essential Oil, Garlic Oil, Geranium Essential Oil, Ginger Essential Oil, Globulus Essential Oil, Grapefruit, Green tea essential oil, Holy Basil Essential Oil, Indian Rosemary Essential Oil, Jojoba Oil, Juniper Berry Oil, Karanj Oil, Lavender Oil, Lemongrass Essential Oil, Lemongrass Essential Oil, Lemongrass Essential Oil, Lime Oil, Mandarine, Marigold Essential Oil, Mentha arvensis Essential Oil, Mentha citrata Essential Oil, Mentha Peperata Essential Oil, Mentha Spicata Essential Oil, Moringa Seed oil, Nagarmotha Essential oil, Neem Oil, Nutmeg Essential Oil, Palmarosa Essential Oil, Patchouli Essential Oil, Peanut Oil, Sage Essential Oil, Soybean Oil, Spearmint, Sweet Marjoram oil, Tagetes minuta Essential Oil, Tamannu Essential Oil, Tea tree essential oil, Tea Tree Oil, Vetiver Essential Oil, Orange Oil, Thyme red Essential Oil, Wormwood, or Ylang Ylang Essential Oil.

6. The nanoemulsion composite mixture as set forth in claim 1, wherein the co-surfactants are Benzalkonium chloride, Behentrimonium chloride, Carbethopendecinium bromide, Didecyldimethylammonium chloride, Benzalkonium chloride, Cetalkonium chloride, Dimethyldioctadecylammonium bromide, Benzethonium chloride, Cetrimonium bromide, Dimethyldioctadecylammonium chloride, Benzododecinium bromide, Cetrimonium chloride, Dioleoyl-3-trimethylammonium propane, Bronidox, Cetylpyridinium chloride, Domiphen bromide, Lauryl methyl gluceth-10 hydroxypropyl dimonium chloride, Pahutoxin, Tetramethylammonium hydroxide, Octenidine dihydrochloride, Stearalkonium chloride, Thonzonium bromide, Olaflur, N-Oleyl-1,3-propanediamine, or Octenidine dihydrochloride.

7. A nanoemulsion composite mixture for antimicrobial activities, consisting essentially of:
biodegradable polymers, co-surfactants, surfactants and thickeners together entrapping an oil,
wherein the nanoemulsion composite mixture is dissolved in glacial acetic acid and dichloromethane,
wherein water is used as the vehicle to hold the nanoemulsion composite mixture, and
wherein in the nanoemulsion composite mixture:
the biodegradable polymers are within 5-8%,
the glacial acetic acid is within 0.5-1.5%,
the oil is within 0.5-1.5%,
the surfactant is within 0.5-1.5%,
the thickener is within 15-25%,
the dichloromethane is within 2.5-10%, and
the co-surfactants is within 0.5-1.5%.

8. The nanoemulsion composite mixture as set forth in claim 7, wherein the thickener is glycerin.

9. The nanoemulsion composite mixture as set forth in claim 7, wherein the surfactant is Tween 80.

10. The nanoemulsion composite mixture as set forth in claim 7, wherein the biodegradable polymers are Chitosan, 1,3 Propyleneglycolalginate, Gelatin, Pectin, 3-hydroxypentanoic acid, Glucose, Poly (acrylates), Albumin, Gluten, Poly (alkyl cyanoacrylate), Alginate, Glycerol and various, Poly (e-caprolactone), Alginic acid, Hydroxyethyl starch, poly (lactic-co-glycolic acid), Carrageenan, Hydroxypropylstarch, Polyanhydrides, Casein Cellulose, Lactic acid, Polylactic acid, Cellulose, Lactic acid, butyl ester, Polyphosphazenes, Cellulose acetate, Lignocellulose, polyvinyl alcohol, Cellulose acetate butyrate, Sucrose, Propyleneglycolalginate, Cellulose derivative, zein, starch, Chitin, Dextrin, Edible Starch, Hydrolysed Starch or Dextrin.

11. The nanoemulsion composite mixture as set forth in claim 7, wherein the oil is Green tea essential oil, Ajwain Essential Oil, Almond Oil, Ambrette Seed Essential Oil, Angelica root Oil, Basil Essential oil, Bay oil, Bergamot oil, Black Pepper Oil, Black Seed essential oil, Cade oil, Cajeput oil, Calamus Essential Oil, Caraway seed, Carrot Seed Oil, Cassia Bark Oil, Castor Oil, Chamomile Essential Oil, Cinnamon essential oil, Citronella Essential Oil, Coconut Oil, Coriander Seed Oil, Cumin oil, Curry Leaf Oil, Cypress oil, Davana Essential Oil, Dill Seed Essential Oil, E. citriodora Essential Oil, Sesame oil, Fennel Essential Oil, Fractionated Coconut Oil, Frankincense Essential Oil, Garlic Oil, Geranium Essential Oil, Ginger Essential Oil, Globulus Essential Oil, Grapefruit, Green tea essential oil, Holy Basil Essential Oil, Indian Rosemary Essential Oil, Jojoba Oil, Juniper Berry Oil, Karanj Oil, Lavender Oil, Lemongrass Essential Oil, Lemongrass Essential Oil, Lemongrass Essential Oil, Lime Oil, Mandarine, Marigold Essential Oil, Mentha arvensis Essential Oil, Mentha Citrata Essential Oil, Mentha Peperata Essential Oil, Mentha Spicata Essential Oil, Moringa Seed oil, Nagarmotha Essential oil, Neem Oil, Nutmeg Essential Oil, Palmarosa Essential Oil, Patchouli Essential Oil, Peanut Oil, Sage Essential Oil, Soybean Oil, Spearmint, Sweet Marjoram oil, Tagetes minuta Essential Oil, Tamannu Essential Oil, Tea tree essential oil, Tea Tree Oil, Vetiver Essential Oil, Orange Oil, Thyme red Essential Oil, Wormwood, or Ylang Ylang Essential Oil.

12. The nanoemulsion composite mixture as set forth in claim 7, wherein the co-surfactants are Benzalkonium Behentrimonium chloride, chloride, Carbethopendecinium bromide, Didecyldimethylammonium chloride, Benzalkonium chloride, Cetalkonium chloride, Dimethyldioctadecylammonium bromide, Benzethonium chloride, Cetrimonium bromide, Dimethyldioctadecylammonium chloride, Benzododecinium bromide, Cetrimonium chloride, Dioleoyl-3-trimethylammonium propane, Bronidox, Cetylpyridinium chloride, Domiphen bromide, Lauryl methyl gluceth-10 hydroxypropyl dimonium chloride, Pahutoxin, Tetramethylammonium hydroxide, Octenidine dihydrochloride, Stearalkonium chloride, Thonzonium bromide, Olaflur, N-Oleyl-1,3-propanediamine, or Octenidine dihydrochloride.
